(19) United States
(12) Patent Application Publication
(10) Pub. No.: US 2025/0002640 A1
(43) Pub. Date: Jan. 2, 2025

(54) MOLDED ARTICLES FOR USE WITH DEGREDATION CHEMICALS

(71) Applicant: Eastman Chemical Company, Kingsport, TN (US)

(72) Inventors: Steven Lee Stafford, Gray, TN (US); Bryan Steven Bishop, Gate City, VA (US); Kathleen Jean White, Kingsport, TN (US); Mason Scott Myers, Kingsport, TN (US)

(73) Assignee: Eastman Chemical Company, Kingsport, TN (US)

(21) Appl. No.: 18/681,961

(22) PCT Filed: Aug. 3, 2022
(86) PCT No.: PCT/US2022/039255

§ 371 Date: Feb. 7, 2024

Related U.S. Application Data
(60) Provisional application No. 63/260,210, filed on Aug. 12, 2021.

(51) Int. Cl. C08G 63/199; A45D 34/00; A61M 35/00

(52) U.S. Cl. CPC C08G 63/199; A45D 34/00; A61M 35/10; A45D 2200/05; C08G 2250/00; C08G 2390/00

(57) ABSTRACT

A shaped article comprising a molded component configured to accommodate or to receive a chemical composition that contains at least one degradation chemical, where the molded component is formed of a copolyester composition having high chemical resistance to the degradation chemicals and having a Tg of at least 95° C.

### FIELD OF THE INVENTION

This invention belongs to the field of polymer-based resins useful for forming articles or components of articles intended for contact with chemical compositions that may cause degradation of polymer properties. In one aspect, the articles/components are intended for contact with such chemical compositions that are intended for body contact or such chemical compositions used on articles that are intended for body contact. Plastic articles or components for such articles made using these resin compositions, such as wearable articles, packaging or dispensing devices for cosmetics of personal care products, medical articles for use with body contact substances, or high touch articles that may contact cleaners, detergents or disinfectants, are also provided.

### BACKGROUND OF THE INVENTION

Plastics are a preferred material for making articles/devices that are wearable, intended for high touch, that contain chemical compositions intended for contact with the body, e.g., packaging or delivery devices for such chemical compositions, or that are otherwise likely to come into contact with chemicals that may cause polymer degradation, based on the relative efficiency of molding parts and articles of various shapes and designs. For example, wearable articles that will likely come into contact with such chemical compositions, such as wearable electronics or other high touch articles/devices, are often manufactured by molding plastic parts that form an assembly to produce the device. Similarly, devices used to deliver/store chemical compositions such as compositions intended for body contact, e.g., skin contact, such as jars, tubes, bags, or dispensing devices, are also manufactured by molding plastic into various shaped articles of component parts.

However, when plastics are used in applications where contact with chemicals will occur, there is the potential for degradation (e.g., cracking, crazing, softening, etc.) of the plastic induced by the chemical environment. Some especially aggressive classes of chemicals include ingredients found in products intended for body contact, such as sunscreens, tanning oils, cosmetics, personal care products, aggressive food ingredients. Aggressive chemicals, such as cleaners, detergents or disinfectants, may also be used on wearable or high touch articles. Many plastics are adversely affected by such aggressive chemicals. Thus, there is a need for plastic materials that have resistance to such chemicals, are easily formed into articles, and maintain acceptable physical properties.

It would be beneficial to be able to provide polymer-based resins that can be melt processed and articles made from such compositions that do not have such drawbacks.

### SUMMARY OF THE INVENTION

Surprisingly, it had been discovered that articles molded from certain copolyester plastics have exceptional resistance to aggressive chemical compositions, such as chemicals intended for body contact, e.g., sunscreen, cosmetics, cleaners, detergents, sanitizers and disinfectants, insect repellant, perfume, oils and fats, aggressive food ingredients, and alcohols, and maintain sufficient physical properties required for the intended use of the articles. In embodiments, such articles are useful as containers and/or other components in articles or devices that will have significant contact with chemical compositions intended for body contact in use, or high touch articles that may be contacted with aggressive chemicals. In one aspect, articles configured to receive a chemical composition intended for body contact can be made from compositions of copolyesters that can be prepared having excellent chemical resistance to such chemical compositions and a glass transition temperature (Tg) exceeding 95° C., or 100° C.

Other aggressive classes of chemicals include solvents such as those found in ink formulations that are often stored or delivered via plastic articles. Thus, in another aspect, articles configured to receive other chemical compositions that comprise a degradation chemical, such as solvents found in ink formulations, can be made from compositions of copolyesters that can be prepared having excellent chemical resistance to such chemical compositions and a glass transition temperature (Tg) exceeding 95° C., or 100° C.

It has been discovered that shaped articles configured to receive (or configured to accommodate contact with) a chemical composition comprising a degradation chemical, e.g., chemical compositions intended for body contact or a chemical composition used on high touch articles, can be prepared from copolyester plastic materials that have resistance to the chemical composition and have physical properties similar to or better than molded articles produced from other typically used oil-based engineering thermoplastics. More specifically, these shaped articles are produced from a copolyester composition that retains its physical properties better than the other plastics after exposure to the chemical composition.

In one aspect of the invention, it is directed to a shaped article configured to receive (or configured to accommodate contact with) a chemical composition that comprises one or more degradation chemicals. In embodiments, the shaped article comprises a copolyester composition, wherein the copolyester composition has a Tg of at least 95° C., or at least 100° C., and has at least one of the following properties chosen from: tensile modulus of greater than 1400 MPa as measured according to ASTM D638 using a 3.2 mm thick bar that has been subjected to 50% relative humidity for 40 hours at 23° C.; a notched izod impact strength of greater than 1000 J/m as measured according to ASTM D256 at 23 C using a 3.2 mm thick bar that has been subjected to 50% relative humidity for 40 hours at 23° C.; a tensile stress at yield of at least 40 MPa, measured according to ASTM D638; a transmission of at least 70 measured according to ASTM D1003 using a 3.2 mm plaque after injection molding at a barrel set point of 249° C. and a mold temperature of 80° C.; or an L* color of at least 85, measured according to ASTM E1348 using a 3.2 mm plaque after injection molding with a barrel temperature of 249° C. and a mold temperature of 80° C. In embodiments, the copolyester composition has at least 2, or at least 3 of the listed properties. In embodiments, the chemical composition is intended for body contact or is a chemical composition used on high touch articles.

In embodiments of the invention, the shaped articles or components thereof can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles.

In embodiments of the invention, the shaped article is chosen from opaque articles, transparent articles, see-through articles, thin-walled articles, technical articles (e.g., articles having a complex design), articles having high design specifications, intricate design articles, containers for holding a chemical composition intended for body contact, or other shaped articles configured to receive (or contact) a chemical composition intended for body contact (or a chemical composition used on high touch articles).

In embodiments, the technical articles, articles having high design specifications, and intricate design articles can be chosen from articles that include electrical/electronic components, perfume or cosmetic containers, medical contact devices or containers, or components thereof.

In one embodiment of the injection molded article, the copolyester composition further comprises at least one property chosen from: tensile modulus of greater than 1400 MPa as measured according to ASTM D638 using a 3.2 mm thick bar that has been subjected to 50% relative humidity for 40 hours at 23° C.; a notched izod impact strength of greater than 1000 J/m as measured according to ASTM D256 at 23 C using a 3.2 mm thick bar that has been subjected to 50% relative humidity for 40 hours at 23° C.; a tensile stress at yield of at least 40 MPa, measured according to ASTM D638; a transmission of at least 70 measured according to ASTM D1003 using a 3.2 mm plaque after injection molding at a barrel set point of 249° C. and a mold temperature of 80° C.; a ΔE value of less than 25, using a 3.2 mm plaque after injection molding with a barrel temperature of 249° C. and a mold temperature of 80° C.; or an L* color of at least 85, measured according to ASTM E1348 using a 3.2 mm plaque after injection molding with a barrel temperature of 249° C. and a mold temperature of 80° C. In embodiments, the polymer-based resin comprises at least 2, or at least 3 of the listed properties.

In embodiments in accordance with the various aspects of the invention disclosed herein, the copolyester composition comprises at least one copolyester which comprises:
(a) a dicarboxylic acid component comprising:
   i) 70 to 100 mole % of terephthalic acid residues;
(b) a glycol component comprising:
   i) 5 to 15 mole % of 2,2,4,4-tetramethyl-1,3-cyclobutanediol residues; and
   ii) 85 to 95 mole % of 1,4-cyclohexanedimethanol residues, wherein the total mole % of the dicarboxylic acid component is 100 mole %, and the total mole % of the glycol component is 100 mole %; and wherein the inherent viscosity is 0.60 to 1.2 dL/g as determined in 60/40 (wt/wt) phenol/tetrachloroethane at a concentration of 0.5 g/100 ml at 25° C.; and wherein the polyester has a Tg of 95° C. to 115° C.

In embodiments in accordance with the various aspects of the invention disclosed herein, the copolyester composition comprises at least one copolyester which comprises:
(a) a dicarboxylic acid component comprising:
   i) 70 to 100 mole % of terephthalic acid residues;
(b) a glycol component comprising:
   i) 5 to 15 mole % of 2,2,4,4-tetramethyl-1,3-cyclobutanediol residues; and
   ii) 85 to 95 mole % of 1,4-cyclohexanedimethanol residues, wherein the total mole % of the dicarboxylic acid component is 100 mole %, and the total mole % of the glycol component is 100 mole %; and wherein the inherent viscosity is 0.60 to 1.0 dL/g as determined in 60/40 (wt/wt) phenol/tetrachloroethane at a concentration of 0.5 g/100 ml at 25° C.; and wherein the polyester has a Tg of 95° C. to 115° C.

In embodiments, the dicarboxylic acid component comprises:
i) 95 to 100 mole % of terephthalic acid (TPA) residues; and
ii) 0 to 5 mole % of isophthalic acid (IPA) residues.

In embodiments, the dicarboxylic acid component comprises residues as follows: greater than 95 to 100 mole % TPA and 0 to less than 5 mole % IPA; 96 to 100 mole % TPA and 0 to 4 mole % IPA; 96.5 to 100 mole % TPA and 0 to 3.5 mole % IPA; 97 to 100 mole % TPA and 0 to 3 mole % IPA; 98 to 100 mole % TPA and 0 to 2 mole % IPA; 98.5 to 100 mole % TPA and 0 to 1.5 mole % IPA; 95 to 98.5 mole % TPA and 1.5 to 5 mole % IPA; greater than 95 to 98.5 mole % TPA and 1.5 to less than 5 mole % IPA; 96 to 98.5 mole % TPA and 1.5 to 4 mole % IPA; 96.5 to 98.5 mole % TPA and 1.5 to 3.5 mole % IPA; 97 to 98.5 mole % TPA and 1.5 to 3 mole % IPA; 97.5 to 98.5 mole % TPA and 1.5 to 2.5 mole % IPA; 95 to 98 mole % TPA and 2 to 5 mole % IPA; greater than 95 to 98 mole % TPA and 2 to less than 5 mole % IPA; 96 to 98 mole % TPA and 2 to 4 mole % IPA; 96.5 to 98 mole % TPA and 2 to 3.5 mole % IPA; or 97 to 98 mole % TPA and 2 to 3 mole % IPA.

In embodiments, the glycol component comprises:
i) 7 to 15 mole % of 2,2,4,4-tetramethyl-1,3-cyclobutanediol (TMCD) residues; and
ii) 85 to 93 mole % of 1,4-cyclohexanedimethanol (CHDM) residues.

In embodiments, the glycol component comprises residues as follows: 8 to 15 mole % TMCD and 85 to 92 mole % CHDM; 8 to 14 mole % TMCD and 86 to 92 mole % CHDM; 8 to 13 mole % TMCD and 87 to 92 mole % CHDM; 8 to 12 mole % TMCD and 88 to 92 mole % CHDM; 9 to 15 mole % TMCD and 85 to 91 mole % CHDM; 9 to 14 mole % TMCD and 86 to 91 mole % CHDM; 9 to 13 mole % TMCD and 87 to 91 mole % CHDM; 9 to 12 mole % TMCD and 88 to 91 mole % CHDM; 10 to 15 mole % TMCD and 85 to 90 mole % CHDM; 10 to 14 mole % TMCD and 86 to 90 mole % CHDM; 10 to 13 mole % TMCD and 87 to 90 mole % CHDM; or 10 to 12 mole % TMCD and 88 to 90 mole % CHDM.

In embodiments, the copolyester composition comprises at least one copolyester which comprises:
(a) a dicarboxylic acid component comprising:
   i) 98 to 100 mole %, or 100 mole %, of terephthalic acid residues;
(b) a glycol component comprising:
   i) 10 to 14 mole %, or 11 to 13 mole %, of 2,2,4,4-tetramethyl-1,3-cyclobutanediol residues; and
   ii) 88 to 90 mole %, or 87 to 89 mole %, of 1,4-cyclohexanedimethanol residues,
wherein the total mole % of the dicarboxylic acid component is 100 mole %, and the total mole % of the glycol component is 100 mole %; and wherein the inherent viscosity is 0.70 to 1.0 dL/g, or 0.75 to 0.95 dL/g as determined in 60/40 (wt/wt) phenol/tetrachloroethane at a concentration of 0.5 g/100 ml at 25° C.; and wherein the polyester has a Tg of 100° C. to 115° C.

In embodiments, the copolyester composition comprises at least one copolyester which comprises:
(a) a dicarboxylic acid component comprising:
   i) 97.1 to 98.5 mole %, or 97.3 to 98.3 mole %, of terephthalic acid residues; and
   ii) 1.5 to 2.9 mole %, or 1.7 to 2.7 mole %, of isophthalic acid residues;
(b) a glycol component comprising:
   i) 10 to 12 mole % of 2,2,4,4-tetramethyl-1,3-cyclobutanediol residues; and
   ii) 88 to 90 mole % of 1,4-cyclohexanedimethanol residues,
wherein the total mole % of the dicarboxylic acid component is 100 mole %, and the total mole % of the glycol component is 100 mole %; and wherein the inherent viscosity is 0.70 to 1.0 dL/g, or 0.75 to 0.95 dL/g as determined in 60/40 (wt/wt) phenol/tetrachloroethane at a concentration of 0.5 g/100 ml at 25° C.; and wherein the polyester has a Tg of 100° C. to 115° C. In embodiments, the at least one copolyester is a melt blended copolyester having an IV of 0.70 to 0.90 dL/g, or 0.75 to 0.85 dL/g, or 0.79 to 0.82 dL/g. In embodiments, the melt blended copolyester is solid stated to increase the IV. In embodiments, the solid stated copolyester has an IV from 0.80 to 1.0 dL/g, or 0.85 to 1.0 dL/g, or 0.87 to 0.97 dL/g, or 0.90 to 0.95 dL/g.

In embodiments, the copolyester composition is amorphous. In other embodiments, the copolyester composition is semi-crystalline.

In embodiments, the at least one copolyester is a reactor grade polyester prepared by a process that includes a transesterification reaction of reaction mixture that includes all the monomers for the intended (monomeric) residues to be included in the copolyester. For example, a copolyester intended to include residues of TPA, CHDM and TMCD is prepared by a transesterification reaction that includes each of these monomers. In an embodiment, the reactor grade polyester is amorphous.

In embodiments, the at least one copolyester is a melt blend polyester prepared by a process that includes melt blending at least two different starting polyesters to provide a final copolyester that includes the monomeric residues contained in starting polyesters. For example, a PCTA copolyester containing residues of TPA, IPA and CHDM is melt blended with a PCTM copolyester containing residues of TPA, CHDM and TMCD to provide a final copolyester having residues of TPA, IPA, CHDM and TMCD. In embodiments, the melt blended copolyester has residues in (net) amounts according to any of the embodiments for the copolyester (as described herein).

In embodiments, the melt blended copolyester is subjected to solid stating to increase the inherent viscosity (IV) of the copolyester. In embodiments, the solid stated copolyester has an IV according to any of the embodiments for the copolyester (as described herein).

In embodiments, a system for delivery of a chemical composition that comprises one or more degradation chemicals is provided, the system comprising a shaped article configured to receive the chemical composition and the chemical composition, wherein the shaped article comprises one or more surfaces in contact with the chemical composition and/or configured to contact the chemical composition when the system is used for its intended purpose, and wherein the one or more surfaces are formed from a copolyester composition (as described herein). In embodiments, a majority of the surfaces that are in contact with the chemical composition and/or configured to contact the chemical composition when the system is used for its intended purpose are formed from the copolyester composition. In embodiments, the chemical composition is intended for body contact.

In embodiments, the chemical composition intended for body contact is in the form of a liquid, gel, lotion, paste, mouse, emulsion and/or a dispersion. In embodiments, the chemical composition intended for body contact can be in the form of a spray, e.g., aerosol or pump spray, such as spray on tanning oils or sun screens. In embodiments, the system comprises a shaped article that comprises one or more liquid contact surfaces in contact with a liquid chemical composition intended for body contact and one or more contact surfaces configured to contact a converted form chemical composition (where liquid is converted to another form, e.g., mouse, foam, vapor or atomized mist) intended for body contact when the system is used for its intended purpose. In one embodiment, the one or more liquid contact surfaces and the one or more converted form contact surfaces are in fluid communication and the chemical composition intended for body contact is produced by converting the liquid chemical composition intended for body contact. In one embodiment, the system comprises a shaped article that comprises one or more surfaces in contact with both a liquid chemical composition intended for body contact and a converted form chemical composition.

In embodiments, the system comprises a shaped article that comprises one or more liquid contact surfaces in contact with a liquid chemical composition intended for body contact for at least 5 minutes. In embodiments, the system comprises a shaped article that comprises one or more contact surfaces in contact with a converted form chemical composition intended for body contact repetitively for a total contact time of at least 5 minutes.

In embodiments, the chemical composition intended for body contact comprises a degradation chemical that is present in an amount of at least 1 wt %, or at least 5 wt %, based on the total weight of the chemical composition intended for body contact.

### DETAILED DESCRIPTION

In one aspect of the invention, it is directed to a shaped article configured to receive a chemical composition that comprises one or more degradation chemicals, the article comprising a copolyester composition, wherein the copolyester composition has a Tg of at least 95° C., or at least 100° C., comprises a copolyester (as described herein), and has at least one of the following properties chosen from: tensile modulus of greater than 1400 MPa as measured according to ASTM D638 using a 3.2 mm thick bar that has been subjected to 50% relative humidity for 40 hours at 23° C.; a notched izod impact strength of greater than 1000 J/m as measured according to ASTM D256 at 23 C using a 3.2 mm thick bar that has been subjected to 50% relative humidity for 40 hours at 23° C.; a tensile stress at yield of at least 40 MPa, measured according to ASTM D638; a transmission of at least 70 measured according to ASTM D1003 using a 3.2 mm plaque after injection molding at a barrel set point of 249° C. and a mold temperature of 80° C.; a ΔE value of less than 25, using a 3.2 mm plaque after injection molding with a barrel temperature of 249° C. and a mold temperature of 80° C.; or an L* color of at least 85, measured according to ASTM E1348 using a 3.2 mm plaque after injection molding with a barrel temperature of 249° C. and a mold temperature of 80° C. In embodiments, the polymer-based resin has at least 2, or at least 3 of the listed properties. In one embodiment, the chemical composition is intended for body contact.

In another aspect of the invention, it is directed to a shaped article configured to accommodate contact with a chemical composition intended for body contact (or a chemical composition used on high touch articles) and comprising such a copolyester composition. Such articles can include wearable articles that will likely or inevitably contact one or more chemical compositions intended for skin contact, such as sunscreens, insect repellant, sanitizers, or personal care or cosmetic products. Such articles can include, for example watches, fitness trackers, wrist bands or bracelets, sunglasses, earbuds, or various clothing articles. Such articles can also include high touch articles that will likely or inevitably contact one or more aggressive chemical compositions, such as cleaners, disinfectants, or detergents.

The term “polyester”, as used herein, is intended to include “copolyesters” and is understood to mean a synthetic polymer prepared by the reaction of one or more difunctional carboxylic acids and/or multifunctional carboxylic acids with one or more difunctional hydroxyl compounds and/or multifunctional hydroxyl compounds. Typically, the difunctional carboxylic acid can be a dicarboxylic acid and the difunctional hydroxyl compound can be a dihydric alcohol such as, for example, glycols and diols. The term “glycol” as used in this application includes, but is not limited to, diols, glycols, and/or multifunctional hydroxyl compounds, for example, branching agents. Alternatively, the difunctional carboxylic acid may be a hydroxy carboxylic acid such as, for example, p-hydroxybenzoic acid, and the difunctional hydroxyl compound may be an aromatic nucleus bearing 2 hydroxyl substituents such as, for example, hydroquinone. The term “residue”, as used herein, means any organic structure incorporated into a polymer through a polycondensation and/or an esterification reaction from the corresponding monomer. The term “repeating unit”, as used herein, means an organic structure having a dicarboxylic acid residue and a diol residue bonded through a carbonyloxy group. Thus, for example, the dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, half-esters, salts, half-salts, anhydrides, mixed anhydrides, or mixtures thereof, useful in a reaction process with a diol to make polyester. Furthermore, as used in this application, the term “diacid” includes multifunctional acids, for example, branching agents. As used herein, the term “terephthalic acid” is intended to include terephthalic acid itself and residues thereof as well as any derivative of terephthalic acid, including its associated acid halides, esters, half-esters, salts, half-salts, anhydrides, mixed anhydrides, or mixtures thereof or residues thereof useful in a reaction process with a diol to make polyester.

In one embodiment, terephthalic acid may be used as the starting material. In another embodiment, dimethyl terephthalate may be used as the starting material. In yet another embodiment, mixtures of terephthalic acid and dimethyl terephthalate may be used as the starting material and/or as an intermediate material. In embodiments, at least a portion of the terephthalic acid or dimethyl terephthalate used as a starting material has recycle content derived directly or indirectly from recycle waste. In embodiments, the recycle content can be obtained from waste plastic that contains terephthalic acid residues, e.g., recovered monomers obtained through a solvolysis (e.g., methanolysis) process. In embodiments, the terephthalic acid residues present in the polyester (according to any of the embodiments herein) contains at least 50 mole %, or at least 75 mole %, or 100 mole % recycle content. In embodiments, the dicarboxylic acid component of the polyester comprises monomer residues having at least 50 mole % recycle content, or at least 75 mole % recycle content, or 100 mole % recycle content.

The polyesters used in the present invention typically can be prepared from dicarboxylic acids and diols which react in substantially equal proportions and are incorporated into the polyester polymer as their corresponding residues. The polyesters of the present invention, therefore, can contain substantially equal molar proportions of acid residues (100 mole %) and diol (and/or multifunctional hydroxyl compounds) residues (100 mole %) such that the total moles of repeating units is equal to 100 mole %. The mole percentages provided in the present disclosure, therefore, may be based on the total moles of acid residues, the total moles of diol residues, or the total moles of repeating units. For example, a polyester containing 4 mole % isophthalic acid, based on the total acid residues, means the polyester contains 4 mole % isophthalic acid residues out of a total of 100 mole % acid residues. Thus, there are 4 moles of isophthalic acid residues among every 100 moles of acid residues. In another example, a polyester containing 15 mole % 2,2,4,4-tetramethyl-1,3-cyclobutanediol, based on the total diol residues, means the polyester contains 15 mole % 2,2,4,4-tetramethyl-1,3-cyclobutanediol residues out of a total of 100 mole % diol residues. Thus, there are 15 moles of 2,2,4,4-tetramethyl-1,3-cyclobutanediol residues among every 100 moles of diol residues.

In other aspects of the invention, the Tg of the polyesters useful in the invention can be at least one of the following ranges: 95 to 115° C.; 95 to 110° C.; 95 to 105° C.; 95 to 100° C.; 100 to 115° C.; 100 to 110° C.; 100 to 105° C.; 105 to 115° C.; 105 to 110° C.; and 110 to 115° C.

In other aspects of the invention, the glycol component for the polyesters useful in the invention include but are not limited to at least one of the following combinations of ranges: 5 to 15 mole % 2,2,4,4-tetramethyl-1,3-cyclobutanediol and 85 to 95 mole % 1,4-cyclohexanedimethanol; 5 to 14 mole % 2,2,4,4-tetramethyl-1,3-cyclobutanediol and 86 to 95 mole % 1,4-cyclohexanedimethanol; 5 to 13 mole % 2,2,4,4-tetramethyl-1,3-cyclobutanediol and 87 to 95 mole % 1,4-cyclohexanedimethanol; 5 to 12 mole % 2,2,4,4-tetramethyl-1,3-cyclobutanediol and 88 to 95 mole % 1,4-cyclohexanedimethanol; 5 to 11 mole % 2,2,4,4-tetramethyl-1,3-cyclobutanediol and 89 to 95 mole % 1,4-cyclohexanedimethanol; 6 to 15 mole % 2,2,4,4-tetramethyl-1,3-cyclobutanediol and 85 to 94 mole % 1,4-cyclohexanedimethanol; 6 to 14 mole % 2,2,4,4-tetramethyl-1,3-cyclobutanediol and 86 to 94 mole % 1,4-cyclohexanedimethanol; 6 to 13 mole % 2,2,4,4-tetramethyl-1,3-cyclobutanediol and 87 to 94 mole % 1,4-cyclohexanedimethanol; 6 to 12 mole % 2,2,4,4-tetramethyl-1,3-cyclobutanediol and 88 to 94 mole % 1,4-cyclohexanedimethanol; 6 to 11 mole % 2,2,4,4-tetramethyl-1,3-cyclobutanediol and 89 to 94 mole % 1,4-cyclohexanedimethanol; 7 to 15 mole % 2,2,4,4-tetramethyl-1,3-cyclobutanediol and 85 to 93 mole % 1,4-cyclohexanedimethanol; 7 to 14 mole % 2,2,4,4-tetramethyl-1,3-cyclobutanediol and 86 to 93 mole % 1,4-cyclohexanedimethanol; 7 to 13 mole % 2,2,4,4-tetramethyl-1,3-cyclobutanediol and 87 to 93 mole % 1,4-cyclohexanedimethanol; 7 to 12 mole % 2,2,4,4-tetramethyl-1,3-cyclobutanediol and 88 to 93 mole % 1,4-cyclohexanedimethanol; 7 to 11 mole % 2,2,4,4-tetramethyl-1,3-cyclobutanediol and 89 to 93 mole % 1,4-cyclohexanedimethanol; 8 to 15 mole % 2,2,4,4-tetramethyl-1,3-cyclobutanediol and 85 to 92 mole % 1,4-cyclohexanedimethanol; 8 to 14 mole % 2,2,4,4-tetramethyl-1,3-cyclobutanediol and 86 to 92 mole % 1,4-cyclohexanedimethanol; 8 to 13 mole % 2,2,4,4-tetramethyl-1,3-cyclobutanediol and 87 to 92 mole % 1,4-cyclohexanedimethanol; 8 to 12 mole % 2,2,4,4-tetramethyl-1,3-cyclobutanediol and 88 to 92 mole % 1,4-cyclohexanedimethanol; 8 to 11 mole % 2,2,4,4-tetramethyl-1,3-cyclobutanediol and 89 to 92 mole % 1,4-cyclohexanedimethanol; 9 to 15 mole % 2,2,4,4-tetramethyl-1,3-cyclobutanediol and 85 to 91 mole % 1,4-cyclohexanedimethanol; 9 to 14 mole % 2,2,4,4-tetramethyl-1,3-cyclobutanediol and 86 to 91 mole % 1,4-cyclohexanedimethanol; 9 to 13 mole % 2,2,4,4-tetramethyl-1,3-cyclobutanediol and 87 to 91 mole % 1,4-cyclohexanedimethanol; 9 to 12 mole % 2,2,4,4-tetramethyl-1,3-cyclobutanediol and 88 to 91 mole % 1,4-cyclohexanedimethanol; 9 to 11 mole % 2,2,4,4-tetramethyl-1,3-cyclobutanediol and 89 to 91 mole % 1,4-cyclohexanedimethanol; 10 to 15 mole % 2,2,4,4-tetramethyl-1,3-cyclobutanediol and 85 to 90 mole % 1,4-cyclohexanedimethanol; 10 to 14 mole % 2,2,4,4-tetramethyl-1,3-cyclobutanediol and 86 to 90 mole % 1,4-cyclohexanedimethanol; 10 to 13 mole % 2,2,4,4-tetramethyl-1,3-cyclobutanediol and 87 to 90 mole % 1,4-cyclohexanedimethanol; 10 to 12 mole % 2,2,4,4-tetramethyl-1,3-cyclobutanediol and 88 to 90 mole % 1,4-cyclohexanedimethanol; and 10 to 11 mole % 2,2,4,4-tetramethyl-1,3-cyclobutanediol and 89 to 90 mole % 1,4-cyclohexanedimethanol.

For certain embodiments of the invention, the polyesters useful in the invention may exhibit at least one of the following inherent viscosities as determined in 60/40 (wt/wt) phenol/tetrachloroethane at a concentration of 0.5 g/100 ml at 25° C.: 0.60 to 1.2 dL/g; 0.60 to 1.1 dL/g; 0.60 to 1 dL/g; 0.60 to less than 1 dL/g; 0.60 to 0.98 dL/g; 0.60 to 0.95 dL/g; 0.60 to 0.90 dL/g; 0.60 to 0.85 dL/g; 0.60 to 0.80 dL/g; 0.60 to 0.75 dL/g; 0.60 to less than 0.75 dL/g; 0.60 to 0.72 dL/g; 0.60 to 0.70 dL/g; 0.60 to less than 0.70 dL/g; 0.60 to 0.68 dL/g; 0.60 to less than 0.68 dL/g; 0.60 to 0.65 dL/g; 0.65 to 1.2 dL/g; 0.65 to 1.1 dL/g; 0.65 to 1 dL/g; 0.65 to less than 1 dL/g; 0.65 to 0.98 dL/g; 0.65 to 0.95 dL/g; 0.65 to 0.90 dL/g; 0.65 to 0.85 dL/g; 0.65 to 0.80 dL/g; 0.65 to 0.75 dL/g; 0.65 to less than 0.75 dL/g; 0.65 to 0.72 dL/g; 0.65 to 0.70 dL/g; or 0.65 to less than 0.70 dL/g; 0.70 to 1.2 dL/g; 0.70 to 1.1 dL/g; 0.70 to 1 dL/g; 0.70 to less than 1 dL/g; 0.70 to 0.98 dL/g; 0.70 to 0.95 dL/g; 0.70 to 0.90 dL/g; 0.70 to 0.85 dL/g; 0.70 to 0.80 dL/g; 0.70 to 0.75 dL/g; 0.70 to less than 0.75 dL/g; 0.75 to 1.2 dL/g; 0.75 to 1.1 dL/g; 0.75 to 1 dL/g; 0.75 to less than 1 dL/g; 0.75 to 0.98 dL/g; 0.75 to 0.95 dL/g; 0.75 to 0.90 dL/g; 0.75 to 0.85 dL/g; 0.75 to 0.80 dL/g; 0.75 to less than 0.80 dL/g; 0.80 to 1.2 dL/g; 0.80 to 1.1 dL/g; 0.80 to 1 dL/g; 0.80 to less than 1 dL/g; 0.80 to 0.98 dL/g; 0.80 to 0.95 dL/g; 0.80 to 0.90 dL/g; 0.80 to 0.85 dL/g; 0.80 to less than 0.85 dL/g; 0.85 to 1.2 dL/g; 0.85 to 1.1 dL/g; 0.85 to 1 dL/g; 0.85 to less than 1 dL/g; 0.85 to 0.98 dL/g; 0.85 to 0.95 dL/g; 0.85 to 0.90 dL/g; 0.85 to less than 0.90 dL/g; 0.90 to 1.2 dL/g; 0.90 to 1.1 dL/g; 0.90 to 1 dL/g; 0.90 to less than 1 dL/g; 0.90 to 0.98 dL/g; 0.90 to 0.95 dL/g; or 0.90 to less than 0.95 dL/g. It is contemplated that the polyester compositions of the invention can possess at least one of the inherent viscosity ranges described herein and at least one of the monomer ranges for the compositions described herein unless otherwise stated. It is also contemplated that the polyester compositions of the invention can possess at least one of the Tg ranges described herein and at least one of the monomer ranges for the compositions described herein unless otherwise stated. It is also contemplated that the polyester compositions of the invention can possess at least one of the Tg ranges described herein, at least one of the inherent viscosity ranges described herein, and at least one of the monomer ranges for the compositions described herein unless otherwise stated.

For the desired polyester, the molar ratio of cis/trans 2,2,4,4-tetramethyl-1,3-cyclobutanediol can vary from the pure form of each or mixtures thereof. In certain embodiments, the molar percentages for cis and/or trans 2,2,4,4-tetramethyl-1,3-cyclobutanediol are greater than 50 mole % cis and less than 50 mole % trans; or greater than 55 mole % cis and less than 45 mole % trans; or 30 to 70 mole % cis and 70 to 30% trans; or 40 to 60 mole % cis and 60 to 40 mole % trans; or 50 to 70 mole % trans and 50 to 30 mole % cis; or 50 to 70 mole % cis and 50 to 30% trans or 60 to 70 mole % cis and 30 to 40 mole % trans; or greater than 70 mole % cis and less than 30 mole % trans; wherein the total sum of the mole percentages for cis- and trans-2,2,4,4-tetramethyl-1,3-cyclobutanediol is equal to 100 mole %. The molar ratio of cis/trans 1,4-cyclohexandimethanol can vary within the range of 50/50 to 0/100, for example, between 40/60 to 20/80. The cis/trans ratio of the compositions can determined by proton nuclear magnetic resonance (NMR) spectroscopy.

In certain embodiments, terephthalic acid, or an ester thereof, such as, for example, dimethyl terephthalate, or a mixture of terephthalic acid and an ester thereof, makes up most or all of the dicarboxylic acid component used to form the polyesters useful in the invention. In certain embodiments, terephthalic acid residues can make up a portion or all of the dicarboxylic acid component used to form the present polyester at a concentration of at least 70 mole %, such as at least 80 mole %, at least 90 mole %, at least 95 mole %, at least 99 mole %, or, in one preferred embodiment (e.g., reactor grade), 100 mole %. In certain embodiments, polyesters with higher amounts of terephthalic acid can be used in order to produce higher impact strength properties. For purposes of this disclosure, the terms “terephthalic acid” and “dimethyl terephthalate are used interchangeably herein. In one embodiment, dimethyl terephthalate is part or all of the dicarboxylic acid component used to make the polyesters useful in the present invention. In all embodiments, ranges of from 70 to 100 mole %; or 80 to 100 mole %; or 90 to 100 mole %; or 99 to 100 mole %; or 100 mole % terephthalic acid and/or dimethyl terephthalate and/or mixtures thereof may be used.

In certain embodiments, in addition to terephthalic acid residues, the dicarboxylic acid component of the polyesters useful in the invention can comprise up to 30 mole %, up to 20 mole %, up to 10 mole %, up to 5 mole %, or less than 5 mole %, or up to 3 mole %, or up to 1 mole % of one or more modifying aromatic dicarboxylic acids. In one preferred embodiment, the polyester contains 0 mole % modifying aromatic dicarboxylic acids. Thus, if present, it is contemplated that the amount of one or more modifying aromatic dicarboxylic acids can range from any of these preceding endpoint values including, for example, from 0.01 to 30 mole %, from 0.01 to 20 mole %, from 0.01 to 10 mole %, from 0.01 to 5 mole %, from 0.01 to less than 5 mole %, from 0.01 to 4 mole %, from 0.01 to 3 mole %, from 0.01 to 2 mole %, or from 0.01 to 1 mole % of one or more modifying aromatic dicarboxylic acids. In certain embodiments, the amount of one or more modifying aromatic dicarboxylic acids can range from 1 to 5 mole %, from 1 to less than 5 mole %, from 1 to 4 mole %, from 1 to 3 mole %, from 1 to 2 mole %, or from 1.5 to 5 mole %, from 1.5 to less than 5 mole %, from 1.5 to 4 mole %, from 1.5 to 3.5 mole %, from 1.5 to 3 mole %, from 1.5 to 2.5 mole %, from 1.5 to 2 mole %, or from 2 to 5 mole %, from 2 to less than 5 mole %, from 2 to 4 mole %, from 2 to 3.5 mole %, from 2 to 3 mole %, from 2 to 2.5 mole %, or from 2.5 to 5 mole %, from 2.5 to less than 5 mole %, from 2.5 to 4 mole %, from 2.5 to 3.5 mole %, from 2.5 to 3 mole %, or from 3 to 5 mole %, from 3 to less than 5 mole %, from 3 to 4 mole %, from 3 to 3.5 mole %, or from 3.5 to 5 mole %, from 3.5 to less than 5 mole %, from 3.5 to 4 mole %, from 4 to 5 mole %, from 4 to less than 5 mole %, of one or more modifying aromatic dicarboxylic acids.

In one embodiment, modifying aromatic dicarboxylic acids that may be used in the present invention include but are not limited to those having up to 20 carbon atoms, and that can be linear, para-oriented, or symmetrical. Examples of modifying aromatic dicarboxylic acids which may be used in this invention include, but are not limited to, isophthalic acid, 4,4′-biphenyldicarboxylic acid, 1,4-, 1,5-, 2,6-, 2,7-naphthalenedicarboxylic acid, and trans-4,4′-stilbenedicarboxylic acid, and esters thereof. In one embodiment, isophthalic acid is the modifying aromatic dicarboxylic acid. The preferred embodiment of the invention is for 100% of the dicarboxylic acid component based on terephthalic acid residues.

The carboxylic acid component of the polyesters useful in the invention can be further modified with up to 10 mole %, such as up to 5 mole % or up to 1 mole % of one or more aliphatic dicarboxylic acids containing 2-16 carbon atoms, such as, for example, malonic, succinic, glutaric, adipic, pimelic, suberic, azelaic and dodecanedioic dicarboxylic acids. Certain embodiments can also comprise 0.01 or more mole %, such as 0.1 or more mole %, 1 or more mole %, 5 or more mole %, or 10 or more mole % of one or more modifying aliphatic dicarboxylic acids. In one preferred embodiment, the polyester contains 0 mole % modifying aliphatic dicarboxylic acids. Thus, if present, it is contemplated that the amount of one or more modifying aliphatic dicarboxylic acids can range from any of these preceding endpoint values including, for example, from 0.01 to 10 mole % and from 0.1 to 10 mole %. The total mole % of the dicarboxylic acid component is 100 mole %.

Esters of terephthalic acid and the other modifying dicarboxylic acids or their corresponding esters and/or salts may be used instead of the dicarboxylic acids. Suitable examples of dicarboxylic acid esters include, but are not limited to, the dimethyl, diethyl, dipropyl, diisopropyl, dibutyl, and diphenyl esters. In one embodiment, the esters are chosen from at least one of the following: methyl, ethyl, propyl, isopropyl, and phenyl esters.

The 1,4-cyclohexanedimethanol may be cis, trans, or a mixture thereof, for example, a cis/trans ratio of 60:40 to 40:60. In another embodiment, the trans-1,4-cyclohexanedimethanol can be present in the amount of 60 to 80 mole %.

The glycol component of the polyester portion of the polyester compositions useful in the invention can contain 14 mole % or less of one or more modifying glycols which are not 2,2,4,4-tetramethyl-1,3-cyclobutanediol or 1,4-cyclohexanedimethanol; in another embodiment, the polyesters useful in the invention can contain 10 mole % or less of one or more modifying glycols. In another embodiment, the polyesters useful in the invention can contain 5 mole % or less of one or more modifying glycols. In another embodiment, the polyesters useful in the invention can contain 3 mole % or less of one or more modifying glycols. In the preferred embodiment, the polyesters useful in the invention may contain 0 mole % modifying glycols. Certain embodiments can also contain 0.01 or more mole %, such as 0.1 or more mole %, 1 or more mole %, 5 or more mole %, or 10 or more mole % of one or more modifying glycols. Thus, if present, it is contemplated that the amount of one or more modifying glycols can range from any of these preceding endpoint values including, for example, from 0.1 to 10 mole %.

Modifying glycols useful in the polyesters useful in the invention refer to diols other than 2,2,4,4-tetramethyl-1,3-cyclobutanediol and 1,4-cyclohexanedimethanol and may contain 2 to 16 carbon atoms. Examples of suitable modifying glycols include, but are not limited to, ethylene glycol, diethylene glycol, 1,2-propanediol, 1,3-propanediol, neopentyl glycol, 1,4-butanediol, 1,5-pentanediol, 1,6-hexanediol, p-xylene glycol or mixtures thereof. In one embodiment, the modifying glycol is ethylene glycol. In another embodiment, the modifying glycols include but are not limited to 1,3-propanediol and/or 1,4-butanediol. In another embodiment, ethylene glycol is excluded as a modifying diol. In another embodiment, 1,3-propanediol and 1,4-butanediol are excluded as modifying diols. In another embodiment, 2,2-dimethyl-1,3-propanediol is excluded as a modifying diol. The polyesters useful the invention can comprise from 0 to 10 mole percent, for example, from 0.01 to 5 mole percent, from 0.01 to 1 mole percent, from 0.05 to 5 mole percent, from 0.05 to 1 mole percent, or from 0.1 to 0.7 mole percent, or 0.1 to 0.5 mole percent, based the total mole percentages of either the diol or diacid residues; respectively, of one or more residues of a branching monomer, also referred to herein as a branching agent, having 3 or more carboxyl substituents, hydroxyl substituents, or a combination thereof. In certain embodiments, the branching monomer or agent may be added prior to and/or during and/or after the polymerization of the polyester. The polyester(s) useful in the invention can thus be linear or branched. In certain embodiments, the branching monomer or agent may be added prior to and/or during and/or after the polymerization.

Examples of branching monomers include, but are not limited to, multifunctional acids or multifunctional alcohols such as trimellitic acid, trimellitic anhydride, pyromellitic dianhydride, trimethylolpropane, glycerol, pentaerythritol, citric acid, tartaric acid, 3-hydroxyglutaric acid and the like. In one embodiment, the branching monomer residues can comprise 0.1 to 0.7 mole percent of one or more residues chosen from at least one of the following: trimellitic anhydride, pyromellitic dianhydride, glycerol, sorbitol, 1,2,6-hexanetriol, pentaerythritol, trimethylolethane, and/or trimesic acid. The branching monomer may be added to the polyester reaction mixture or blended with the polyester in the form of a concentrate as described, for example, in U.S. Pat. Nos. 5,654,347 and 5,696,176, whose disclosure regarding branching monomers is incorporated herein by reference.

The polyesters useful in the invention can be made by processes known from the literature such as, for example, by processes in homogenous solution, by transesterification processes in the melt, and by two phase interfacial processes. Suitable methods include, but are not limited to, the steps of reacting one or more dicarboxylic acids with one or more glycols at a temperature of 100° C. to 315° C. at a pressure of 0.1 to 760 mm Hg for a time sufficient to form a polyester. See U.S. Pat. No. 3,772,405 for methods of producing polyesters, the disclosure regarding such methods is hereby incorporated herein by reference.

The polyesters useful in this invention can also be prepared by reactive melt blending and extrusion of two polyesters. For example: a polyester containing 100% terephthalic acid residues; 10 mole % 2,2,4,4-tetramethyl-1,3-cyclobutanediol residues, and 90 mole % 1,4-cyclohexanedimethanol can be prepared by reactive melt blending and extrusion of equal amounts of a polyester containing 100 mole % terephthalic residues and 100% 1,4-cyclohexanedimethanol with another polyester containing 100 mole % terephthalic residues; 80 mole % 1,4-cyclohexanedimethanol residues, and 20 mole % 2,2,4,4-tetramethyl-1,3-cyclobutanediol residues.

In embodiments, the polyesters of this invention, prepared in a reactor or by melt blending/extrusion, can subsequently be crystallized if needed and solid stated by techniques known in the art to further increase the IV.

In embodiments, the article made from copolyester composition can be amorphous. For purposes of this disclosure, amorphous means a crystallinity or less than 1%. In other embodiments, the article made from copolyester composition can be semi-crystalline, e.g., by crystallizing with heat. In embodiments, the article of the invention has a crystallinity of from 1 to 40%, or 1 to 35%, or 1 to 30%, or 5 to 40%, or 5 to 35%, or 5 to 30%, or 10 to 40%, or 10 to 35%, or 10 to 30%.

In other embodiments, the article made from the copolyester composition can have strain induced crystallinity. Strain induced crystallization refers to a phenomenon in which an initially amorphous solid material undergoes a phase transformation in which some amorphous domains are converted to crystalline domains due to the application of strain. This phenomenon has important effects in strength and fatigue properties.

In embodiments, the article of the invention has a strain induced crystallinity of from 1 to 40%, or 1 to 35%, or 1 to 30%, or 5 to 40%, or 5 to 35%, or 5 to 30%, or 10 to 40%, or 10 to 35%, or 10 to 30%, when stretched at a temperature above the Tg of the polyester, e.g., during molding or forming processes, such as stretch blow molding.

In embodiments, the article is a clear semi-crystalline article comprising a copolyester that has a crystallization half-time of less than 10 minutes but greater than about 30 seconds. In embodiments, the copolyester has a crystallization half-time from 30 seconds to 5 minutes, or 30 seconds to 3 minutes, or 30 seconds to 2 minutes, or 30 seconds to 1.5 minutes.

In embodiments, the article of the invention can comprise the polyester of the invention having a melting temperature (Tm) from 260° C. to 300° C.

In addition, the polyester useful in this invention may also contain from 0.01 to 25% by weight or 0.01 to 20% by weight or 0.01 to 15% by weight or 0.01 to 10% by weight or 0.01 to 5% by weight of the total weight of the polyester composition of common additives such as colorants, dyes, mold release agents, reheat additives, flame retardants, plasticizers, stabilizers, including but not limited to, UV stabilizers, thermal stabilizers and/or reaction products thereof, fillers, and impact modifiers. Examples of typical commercially available impact modifiers well known in the art and useful in this invention include, but are not limited to, ethylene/propylene terpolymers; functionalized polyolefins, such as those containing methyl acrylate and/or glycidyl methacrylate; styrene-based block copolymeric impact modifiers; and various acrylic core/shell type impact modifiers. For example, UV additives can be incorporated into articles of manufacture through addition to the bulk, through application of a hard coat, or through coextrusion of a cap layer. Residues of such additives are also contemplated as part of the polyester composition.

The polyesters useful in the invention can comprise at least one chain extender. Suitable chain extenders include, but are not limited to, multifunctional (including, but not limited to, bifunctional) isocyanates, multifunctional epoxides, including for example, epoxylated novolacs, and phenoxy resins. In certain embodiments, chain extenders may be added at the end of the polymerization process or after the polymerization process. If added after the polymerization process, chain extenders can be incorporated by compounding or by addition during conversion processes such as injection molding or extrusion. The amount of chain extender used can vary depending on the specific monomer composition used and the physical properties desired but is generally about 0.1 percent by weight to about 10 percent by weight, preferably about 0.1 to about 5 percent by weight, based on the total weight of the polyester.

Thermal stabilizers are compounds that stabilize polyesters during polyester manufacture and/or post polymerization including, but not limited to, phosphorous compounds including but not limited to phosphoric acid, phosphorous acid, phosphonic acid, phosphinic acid, phosphonous acid, and various esters and salts thereof. These can be present in the polyester compositions useful in the invention. The esters can be alkyl, branched alkyl, substituted alkyl, difunctional alkyl, alkyl ethers, aryl, and substituted aryl. In one embodiment, the number of ester groups present in the particular phosphorous compound can vary from zero up to the maximum allowable based on the number of hydroxyl groups present on the thermal stabilizer used. The term “thermal stabilizer” is intended to include the reaction products thereof. The term “reaction product” as used in connection with the thermal stabilizers of the invention refers to any product of a polycondensation or esterification reaction between the thermal stabilizer and any of the monomers used in making the polyester as well as the product of a polycondensation or esterification reaction between the catalyst and any other type of additive.

Reinforcing materials may be useful in the compositions of this invention. The reinforcing materials may include, but are not limited to, carbon filaments, silicates, mica, clay, talc, titanium dioxide, Wollastonite, glass flakes, glass beads and fibers, and polymeric fibers and combinations thereof. In one embodiment, the reinforcing materials are glass, such as, fibrous glass filaments, mixtures of glass and talc, glass and mica, and glass and polymeric fibers.

In embodiments, the articles (configured to receive or to accommodate contact with a chemical composition intended for body contact, or a chemical composition used on high touch articles) can include, but are not limited to, injection blow molded articles, injection stretch blow molded articles, extrusion blow molded articles, extrusion stretch blow molded articles, calendered articles, compression molded articles, and solution casted articles. Methods of making the articles of manufacture, include, but are not limited to, extrusion blow molding, extrusion stretch blow molding, injection blow molding, injection stretch blow molding, calendering, compression molding, and solution casting.

In embodiments, the articles (e.g., configured to receive or to accommodate contact with a chemical composition intended for body contact, or a chemical composition used on high touch articles) can include film(s) and/or sheet(s) comprising the polyester compositions that are formed into the articles of the invention. The methods of forming the polyesters into film(s) and/or sheet(s) are well known in the art. Examples of film(s) and/or sheet(s) of the invention including but not limited to extruded film(s) and/or sheet(s), calendered film(s) and/or sheet(s), compression molded film(s) and/or sheet(s), solution casted film(s) and/or sheet(s). Methods of making film and/or sheet include but are not limited to extrusion, calendering, compression molding, and solution casting.

In embodiments of the invention, the copolyester composition has a notched izod impact strength of at least 800 J/m, or at least 900 J/m, as measured according to ASTM D256 using a 3.2 mm thick bar hat has been subjected to 50% relative humidity for 48 hours at 23° C. In certain embodiments, the polymer-based resin has a notched izod impact strength of at least 1000 J/m, or at least 1050 J/m, as measured according to ASTM D256 using a 3.2 mm thick bar that has been subjected to 50% relative humidity for 48 hours at 23° C.

In embodiments of the invention, the polymer-based resin has a ΔE value of less than 25, or less than 20, or less than 15, or less than 14, or less than 13, or less than 12, or less than 11, or less than 10, or less than 9, or less than 8, or less than 7, or less than 6, or less than 5, using a 3.2 mm plaque after injection molding with a barrel temperature of 249° C. and a mold temperature of 80° C., wherein ΔE is determined by the following equation: ((L*−100)²+(a*−0)²+(b*−0)²)^{1/2}, where the L*, a*, and b* color components were measured according to ASTM E1348. In certain embodiments, the polymer-based resin has a ΔE value in the range from 2 to 25, or from 2 to 20, or from 2 to 15, or from 2 to 14, or from 2 to 13, or from 2 to 12, or from 2 to 11, or from 2 to 10, or from 2 to 9, or from 2 to 8, or from 2 to 7, or from 2 to 6, or from 2 to 5, using a 3.2 mm plaque after injection molding with a barrel temperature of 249° C. and a mold temperature of 80° C., wherein ΔE is determined by the following equation: ((L*−100)²+(a*−0)²+(b*−0)²)^{1/2}, where the L*, a*, and b* color components were measured according to ASTM E1348.

In embodiments of the invention, the polymer-based resin has an L* color of at least 85, or at least 86, or at least 87, or at least 88, or at least 89, or at least 90, or at least 91, or at least 92, or at least 93, or at least 94, or at least 95, measured according to ASTM E1348 using a 3.2 mm plaque after injection molding with a barrel temperature of 249° C. and a mold temperature of 80° C. In certain embodiments, the polymer-based resin has an L* color in the range from 85 to 98, or from 85 to 97, or from 85 to 96, or from 85 to 95, measured according to ASTM E1348 using a 3.2 mm plaque after injection molding with a barrel temperature of 249° C. and a mold temperature of 80° C.

In embodiments of the invention, the polymer-based resin has a b* value is less than 15, or less than 12, or less than 10, or less than 9, or less than 8, or less than 7, or less than 6, or less than 5, or less than 4, measured according to ASTM E1348 using a 3.2 mm plaque after injection molding with a barrel temperature of 249° C. and a mold temperature of 80° C. In certain embodiments, the polymer-based resin has a b* color in the range from 0 to 15, or from 0 to 10, or from 0 to 8, or from 0 to 5, measured according to ASTM E1348 using a 3.2 mm plaque after injection molding with a barrel temperature of 249° C. and a mold temperature of 80° C.

In aspects of this invention, it is directed to shaped articles. In certain embodiments, the shaped articles are not continuously extruded films that are infinite (or continuous) in one direction and fixed in width and thickness in the other two directions, as would be the case in a rolled film. In certain embodiments, a film or sheet can be converted into a shaped article, e.g., by thermoforming into a three-dimensional object, such as a cup or bowl. In embodiments of the invention, the shaped article is not a film or is not a sheet. In embodiments of the invention, the shaped articles can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles.

Shaped articles made from a polyester composition according to the present invention can be shaped via molding or extruding for use in applications for storing (packaging), delivering or accommodating contact with chemical compositions intended for body contact. In embodiments of the invention, the shaped article is chosen from transparent articles, see-through articles, thin-walled articles, technical articles (e.g., articles having a complex design), articles having high design specifications, intricate design articles, containers, wearable articles, household articles, general consumer products, packaging articles, medical articles, high touch articles, or components thereof, where the article is configure to receive or accommodate contact with a chemical composition intended for body contact, or a chemical composition used on high touch articles.

In embodiments, the article is a wearable article or device that will likely contact with chemical compositions intended for body contact that include one or more degradation chemicals. Examples of such wearable articles or devices include fitness trackers, headphones, earbuds, (smart) watches, AR/VR headsets, medical delivery devices, sporting goods (such as sunglasses, helmets, and scuba gear), and cameras.

In embodiments, the article is a medical article or device that will contact chemical compositions intended for body contact or chemical compositions used on such articles or devices (e.g., disinfectants) that include one or more degradation chemicals. Examples of such medical articles or devices include insulin delivery devices, packaging/reservoirs (e.g., configured to receive medicinal compositions, such as CBD oils), farm/veterinary devices (e.g., vaccination devices), drug delivery secondary/shell packaging, fluid drug delivery containers/devices, anesthesia machines, ventilators, blood cassettes, closed system drug transfer devices (e.g., combination pharmaceutical packaging), containers or delivery devices for oncology drugs, or containers for or devices that will be contacted with disinfectants (e.g., alcohols such as IPA, quaternary amines, iodine solution, bleach, and/or hydrogen peroxide/peracetic acid).

In embodiments, the article is a consumer article or device that will contact chemical compositions that include one or more degradation chemicals. Examples of such consumer articles or devices include small appliances (e.g., coffee machine milk/bean tanks, blenders), barware (e.g., restaurant, hotel, resorts, patio, and camping articles that may contact food items having ingredients that include degradation chemicals), laundry containers or articles (e.g., that will/may contact aggressive detergents), water tanks (e.g., that will/may contact fragrant oils such as “apple spice”), food storage containers/serving bowls (that may contact food items having ingredients that include degradation chemicals such as acidic juices/sauces, fats, of oils such as ginger root/oil), food home delivery articles (trays, bins, cups), infant care articles, sports bottles with food infusers, and reusable detergent bottles.

In embodiments, the article is a high touch article or device that will likely contact chemical compositions used on such articles or devices (e.g., disinfectants) that include one or more degradation chemicals. Examples of such high touch articles or devices include toys, protective cases, portable devices (e.g., smartphones, laptops, tablets), computer peripherals (e.g., mice, keyboards, and printers/scanners), power tools, electronic personal care devices (e.g., toothbrushes or hairdryers), point of sale devices (e.g., barcode scanners, card readers, and mobile pay devices), audio/video equipment (e.g., TVs or (smart) speakers), security cameras, digital signage, light switches (high touch areas), smart plugs/lights, networking equipment (e.g., routers or modems), power adaptors/supply devices, and gaming consoles/controllers/electronic toys.

In embodiments, the article or device is intended to be in contact chemical compositions that contain aggressive solvents, such as those used in ink formulations. Examples of such articles/devices include writing devices that contain ink formulations, or ink reservoirs or cartridges for printers. Examples of degradation chemicals that may be included in ink formulations include lactam ring compounds (e.g., 2-pyrrolidone and 1-(2-Hydroxyethyl)-2-pyrrolidone (HE2P)), polylols (e.g., 1,2-butanediol; 1,2-hexanediol; ethylhydroxy-propanediol (EHPD); glycerol; and Dowanol), ketones (e.g., methyl ethyl ketone (MEK)), and alcohols (e.g., ethanol).

In certain embodiments, the polyester composition can be primary molded into forms such as pellets, plates, or parisons, and can then be secondary molded into articles, e.g., conduits, tubes, thin-wall vessels, or thick-wall vessels, configured to receive a chemical composition intended for body contact or a chemical composition used on high touch articles.

The methods of forming the polyester compositions into films, molded articles, and sheeting can be according to methods known in the art. In embodiments, the polyester composition can be over molded onto itself or a different polyester composition and retain an interface bond (or weld line) strength that will not separate (or delaminate) when an article (having such an over mold interface) is used for its intended purpose. In embodiments, transparent polyesters and translucent (or opaque) polyesters can be over molded onto the other. In embodiments, the different polyesters all fall with one or more embodiments of the invention (as discussed herein).

In one aspect, an article or device is provided that comprises a molded component that will likely contact with chemical compositions used on high touch articles/devices, where the molded component is formed of a plastic composition comprising a copolyester composition and having a Tg of at least 95° C.

In one aspect, an article is provided that comprises a molded component configured to receive a chemical composition intended for body contact, where the molded component is formed of a plastic composition comprising a copolyester composition and having a Tg of at least 95° C.

The chemical composition intended for body contact or used on high touch articles contains one or more degradation chemicals in an amount of at least 1 wt %, or at least 5 wt %, or at least 10 wt %, or at least 15 wt % or at least 20 wt %, or at least 25 wt %. Degradation chemical means a chemical that degrades the performance of one or more copolyesters, e.g., CHDM or TMCD containing copolyesters, where the degradation in performance is indicated by at least a 5% reduction in reverse-side impact strength retention after exposure to such chemical, when tested according to the methods disclosed in Examples. In embodiments, the chemical composition intended for body contact contains at least 0.01, or at least 0.05, or at least 0.1, or at least 0.5, or at least 1, or at least 5, or at least 10 wt % of total degradation chemicals.

In embodiments, the degradation chemical is chosen from sunscreen components, insect repellant components, cosmetic components, perfume components, alcohols, glycols, oils, fats, waxes, plant-based oils or extracts, food ingredients, cleaners, disinfectants, detergents, or combinations thereof. In embodiments, sunscreen components can include UV absorbers, such as, for example oxybenzone, avobenzone, octisalate, octocrylene, homosalate, octinoxate, of combinations thereof. In embodiments, insect repellant components can include repellant actives, such as N,N-diethyl-meta-toluamide (DEET), citronella, picardin, plant oils or extracts having repellant properties, or combinations thereof. In embodiments, cosmetic components can include alcohols, glycols, amines, hydroxy acids, oils, fats, waxes, glycerine, colorants, fragrances, or combinations thereof. In embodiments, perfume components can include solvents, alcohols, glycols, hydroxy acids, oils, fragrances, or combinations thereof.

In embodiments, the degradation chemical is a plant-based oil. Plant-based oil means a type of oil that can be found in or obtained from plants. The definition of plants is not to be limited and can include any type or classification of plants, including vascular, non-vascular, seed bearing, spore bearing, angiosperms, and gymnosperms. Plants can include small plants, bushes, or trees. In embodiments, the terpene containing plant-based oil can be synthesized or made without the oil actually being derived from plants, as long as the oil is of a type that can be found in or obtained from plants.

In embodiments, the plant-based oil is a type found primarily in the leaves or flowers of a plant. In embodiments, the plant-based oil is a type found primarily in the seeds or fruit of a plant. In embodiments, the chemical composition intended for body contact can be a combination (e.g., mixture or blend) of different plant-based oils.

In embodiments, the chemical composition intended for body contact comprises a plant-based oil. In embodiments, the plant-based oil is a botanical oil. Botanical oil means an oil of a type obtained from plants that are fatty, dense and non-volatile. In embodiments, the botanical oil is extracted from the root, stem/bark, leaves, flowers, seeds or fruit of a plant, tree or shrub. In embodiments, the botanical oil is cold pressed or extracted by heat. Examples of botanical oils can include rosehip oil (Rosa canina), evening primrose oil (Oenothera biennis), almond oil (Prunus amygdalus dulcis), calendula oil (Calendula officinalis), MCT oil, olive oil, canola oil, corn oil, vegetable oil, cotton seed oil, safflower oil, sunflower seed oil, soapbark tree oil; and extracts, isolates, or derivatives of the foregoing; and combinations of any of the foregoing.

In embodiments, the plant-based oil is an essential oil. Essential oil means a concentrated and volatile substance extracted from plants chosen from aromatic herbs or aromatic plants, where essential refers to an oil that carries a distinctive scent (or essence) of such a plant. Examples of essential oils can include agar oil or oodh, aiwain oil, angelica root oil, anise oil, asafetida oil, balsam of peru, basil oil, bay oil, bergamot oil, black pepper oil, buchu oil, birch oil, camphor oil, cannabis flower essential oil, calamodin oil or calamansi essential oil, caraway seed oil, cardamom seed oil, carrot seed oil, cedar oil, chamomile oil, calamus oil, cinnamon oil, cistus ladanifer, citron oil, citronella oil, clary sage oil, coconut oil, clove oil, coffee oil, coriander oil, costmary oil, costus root oil, cranberry seed oil, cubeb oil, cumin seed oil or black seed oil, cypress oil, cypriol oil, curry leaf oil, davana oil, dill oil, elecampane oil, elemi oil, eucalyptus oil, fennel seed oil, fenugreek oil, fir oil, frankincense oil, galangal oil, galbanum oil, garlic oil, geranium oil, ginger oil, goldenrod oil, grapefruit oil, henna oil, Helichrysum oil, hickory nut oil, horseradish oil, hyssop, Idaho-grown tansy, jasmine oil, juniper berry oil, Laurus nobilis, lavender oil, ledum oil, lemon oil, lemongrass oil, lime oil, Listea cubeba oil, linalool oil, mandarin oil, marjoram oil, melissa oil or lemon balm, Mentha arvensis oil or mint oil, moringa oil, mountain savory oil, mugwort oil, mustard oil, myrrh oil, myrtle oil, neem oil, neroli oil, nutmeg oil, orange oil, oregano oil, orris oil, palo santo oil, parsley oil, patchouli oil, perilla essential oil, pennyroyal oil, peppermint oil, petitgrain oil, pine oil, ravensara oil, red cedar oil, romain chamomile oil, rose oil, rosehip oil, rosemary oil, rosewood oil, sage oil, sandalwood oil, sassafras oil, savory oil, Schisandra oil, spearmint oil, spikenard oil, spruce oil, star anise oil, tangerine oil, tarragon oil, tea tree oil, thyme oil, tsuga oil, turmeric oil, warionia oil, vetiver oil, western red cedar oil, wintergreen oil, yarrow oil, ylang-ylang oil; and extracts, isolates, or derivatives of the foregoing; and combinations of any of the foregoing. In embodiments, the extract, isolate or derivative of the essential oil comprises a terpene or a flavonoid. In embodiments, the terpene is chosen from d-limonene, geraniol, b-pinene, myrcene, terpinolene, or mixtures thereof.

In embodiments, the plant-based oil can be a combination of one or more botanical oils and one or more essential oils. In embodiments, the chemical composition intended for body contact comprises a terpene containing plant-based oil component, where the terpene containing plant-based oil component comprises one or more terpene containing plant-based oils chosen from a botanical oil, an essential oil, or combinations of botanical and essential oils. Examples of plant-based oils include eucalyptus oil, lavender oil, neroli oil, cannabis oil, hemp oil, cannabidiol oil, peppermint oil, sweet orange oil, tea tree oil, lemon oil, lime oil, orange oil; and extracts, isolates, or derivatives of the foregoing oils and/or their plant source; and combinations of any of the foregoing.

In embodiments, the chemical composition (e.g., chemical composition intended for body contact or used on high touch articles) comprises one or more additives chosen from solvents, dispersants, stabilizers, emulsifiers, carriers, solvents, actives. In embodiments, the additional additive(s) can be chosen from glycols, e.g., propylene glycol, glycerin, e.g., plant glycerin, polysorbates, plant-based alkaloids, e.g., pharmaceutical actives, or combinations thereof.

In embodiments, the copolyester composition forming the injection molded article is chosen from any of the copolyester compositions discussed herein. In one embodiment, the copolyester composition comprises at least one copolyester that comprises:
(a) a dicarboxylic acid component comprising:
   i) 96 to 100 mole % of terephthalic acid residues; and
   ii) 0 to 4 mole % of isophthalic acid residues;
(b) a glycol component comprising:
   i) 10 to 15 mole % of 2,2,4,4-tetramethyl-1,3-cyclobutanediol residues; and
   ii) 85 to 90 mole % of 1,4-cyclohexanedimethanol residues,
wherein the total mole % of the dicarboxylic acid component is 100 mole %, and the total mole % of the glycol component is 100 mole %; and wherein the inherent viscosity is 0.70 to 1.0 dL/g, or 0.75 to 0.95 dL/g as determined in 60/40 (wt/wt) phenol/tetrachloroethane at a concentration of 0.5 g/100 ml at 25° C.; and wherein the polyester has a Tg of 100° C. to 115° C.

Properties disclosed herein requiring a test method can be determined as follows:

### Test Methods

Properties disclosed throughout this application can be determined according to the test methods described herein. Samples were (or can be) evaluated using standard ASTM test methods with any special conditions noted below.

TABLE 1
Test Methods

| PROPERTY | COMMENTS |
|---|---|
| Color, b* | Using 3.2 mm thick plaques. Measured using |
| (ASTM E1348) | Hunter Lab Ultrascan Spectra Colorimeter |
| Color, a* | Using 3.2 mm thick plaques. Measured using |
| (ASTM E1348) | Hunter Lab Ultrascan Spectra Colorimeter |
| Color, L* | Using 3.2 mm thick plaques. Measured using |
| (ASTM E1348) | Hunter Lab Ultrascan Spectra Colorimeter |
| Haze (ASTM D1003) | Using 3.2 mm plaques |
| Transmission | Using 3.2 mm plaques |
| (ASTM D1003) | |
| Izod Notched Impact at | Using a 0.32 mm thick bar that has been |
| 23° C. (ASTM D256) | subjected to 50% relative humidity at 23° C. |
| | for 48 hours. |
| Tg (ASTM D3418) | DSC at 20° C./min. |
| Tensile Modulus | Using a 165 mm × 13 mm × 3.2 mm bar |
| (ASTM D638) | subjected to 50% Relative humidity for 40 |
| | hours at 23° C.. |
| Tensile Stress @ Yield | Using a 165 mm × 13 mm × 3.2 mm bar |
| (ASTM D638) | subjected to 50% Relative humidity for 40 |
| | hours at 23° C.. |

The inherent viscosity of the polyesters was determined in 60/40 (wt/wt) phenol/tetrachloroethane at a concentration of 0.5 g/100 ml at 25° C. (according to ASTM D4603).

The glycol content was determined by proton nuclear magnetic resonance (NMR) spectroscopy. All NMR spectra were recorded on a JEOL Eclipse Plus 600 MHz nuclear magnetic resonance spectrometer using either chloroform-trifluoroacetic acid (70-30 volume/volume). Peak assignments for 2,2,4,4-tetramethyl-1,3-cyclobutanediol resonances were made by comparison to model mono- and dibenzoate esters of 2,2,4,4-tetramethyl-1,3-cyclobutanediol. These model compounds closely approximate the resonance positions found in the polymers.

The crystallization half-time, t_{1/2}, was determined by measuring the light transmission of a sample via a laser and photo detector as a function of time on a temperature controlled hot stage. This measurement was done by exposing the polymers to a temperature, Tₘₐₓ, and then cooling it to the desired temperature. The sample was then held at the desired temperature by a hot stage while transmission measurements were made as a function of time. Initially, the sample was visually clear with high light transmission and became opaque as the sample crystallized. The crystallization half-time was recorded as the time at which the light transmission was halfway between the initial transmission and the final transmission. Tₘₐₓ is defined as the temperature required to melt the crystalline domains of the sample (if crystalline domains are present). The Tₘₐₓ reported in the examples below represents the temperature at which each sample was heated to condition the sample prior to crystallization half time measurement. The Tₘₐₓ temperature is dependent on composition and is typically different for each polyester. For example, PCT may need to be heated to some temperature greater than 290° C. to melt the crystalline domains.

Differential scanning calorimetry (DSC) was performed using TA Instruments Model 2920 with a liquid nitrogen cooling accessory. The sample weight, in the range of 8 to 12 mg, was measured and recorded. Samples were first heated (1^{st} heating scan) from 0 to 320° C. at 20° C./min, followed by cooling to 0° C. at 20° C./min (cooling scan), and then heated again from 0 to 320° C. at 20° C. min. Various thermal parameters were measured and recorded. □H_{cc} (cal/g) is the heat of crystallization measured from the cooling scan. T_{cc} is the crystallization peak temperature on the cooling scan. T_{g} is the glass transition temperature measured from 2^{nd} heating scan. Tₘ is the melting point measured during the 2^{nd} heating scan. □H_{ch1} (cal/g) is the heat of crystallization measured during the 1^{st} heating scan. □Hₘ₁ (cal/g) is the heat of melting measured during the 1^{st} heating scan.

The percent crystallinity formed during cooling is calculated by equation (1), assuming a specific heat of fusion of 29 cal/g (based on unmodified PCT).

$${X}_{c} = \frac{( Δ {H}_{c c} )}{29} \times 100 \qquad ( 1 )$$

The peak temperature in the crystallization exotherm (Toc) occurs at 227° C. for unmodified PCT.

The percentage of strain induced crystallinity (Qc) was determined by equation (2) from the first heating scan of films evaluated in a DSC.

$${X}_{c} = \frac{( Δ {H}_{m 1} - Δ {H}_{C H 1} )}{29} \times 100 \qquad ( 2 )$$

As used herein, the abbreviation “wt” means “weight”.

The following examples further illustrate how the compositions of matter of the invention can be made and evaluated, and are intended to be purely exemplary of the invention and are not intended to limit the scope thereof. Unless indicated otherwise, parts are parts by weight, temperature is in degrees C. or is at room temperature, and pressure is at or near atmospheric.

### EXAMPLES

### Example 1

Melt blend copolyester compositions were prepared from the following starting materials:
1) PCTA 13319 (from Eastman Chemical Company)
2) Copolyester TX1000 (from Eastman Chemical Company)
3) Blue Toner Concentrate

The starting materials were melt-blended on a single screw extruder set at 285 C after drying the PCTA 13319 at 120 C and TX1000 at 90 C for 6-8 hrs. in a desiccant bed drying system. The three components were added to the extruder from weight loss feeders at the following concentrations: 49.26 wt % PCTA, 49.44 wt % TX1000 and 1.30 wt % Toner. The resulting (extruded) strand was quenched and cut into cylindrical pellets with a weight average of 0.80 gms/50 pellets. The pellets were amorphous and had an inherent viscosity (IV) of 0.79 to 0.82 (Ex 1-A).

The composition of the Ex 1-A base copolyester had diacid residues of about 97.8 mole % TPA and 2.2 mole % IPA, and glycol residues of about 98.8 mole % CHDM and 11.2 mole % of TMCD. Ex 1-A had a Tg of about 102 C, a Tm of 253 to 259 C and a crystallization half time of about 1 minute at 175 C.

Some of the Ex 1-A amorphous pellets were crystallized at 180 C in a rotating reactor for about 120-180 minutes before increasing the temperature to 225 C for a time sufficient to solid state the copolyester to advance the IV to approximately 0.92 dL/g (Ex 1-B).

### Example 2

### Test Bar Production

Pellets of each copolyester material from Example 1 (EX 1-A and EX 1-B) were injection molded to form standard test bars 0.5 inch×5 inch×0.125 inch (1.27 cm×12.7 cm×0.3 cm). The pellets were molded in A 110 Ton Toyo injection molding machine with barrel capacity 3.4 oz. The copolyester material was injection molded at 1 in/sec injection speed into four test bars per shot with barrel temperature nominally of about 249° C. (480° F.) and mold temperature of about 80° C.

### Test Results

### ESCR—Property Retention in Reverse Side Impact

Testing was conducted using injection molded flex bars with length, width, and thickness of 5.0″, 0.5″, and 0.125″, respectively. Bars were conditioned at 23° C./50% RH for a minimum of 72 hr. Bars were clamped into a constant strain fixture or a 3-point bend fixture at 1.5% strain and exposed to test chemical using a cotton pad saturated with the test chemical, where the pad was placed on the top surface of the bar. The pad was soaked in liquid and the excess squeezed out, or if viscous, applied to the pad as a thin film with a wooden tongue depressor. After the test chemicals were applied to the bars on the side without ejector pin marks, the strain fixtures with bars attached were sealed in polyethylene bags for 24 hours at nominal temperature of 23° C., after which the bars were wiped clean and removed from the strain fixture.

After exposure, the bars were tested at 23° C. for reverse-side impact. The test apparatus was a CEAST Pendulum Impact Tester equipped with a 15-Joule hammer. Bars were positioned in a 2-inch span fixture, with the non-chemically exposed side facing the hammer. Control bars (exposed to water) were impact tested in addition to bars that were exposed to the test chemicals. The comparison of results between the controls and the chemically exposed bars was used to calculate percent retention of original impact energy. The test was repeated five times and the results are an average of the five tests. The results are shown below as follows: Table 2—Personal Care/Cosmetic Chemicals; Table 3—Chemicals Contacting Wearable Articles; and Table 4—Miscellaneous Additional Chemicals.

TABLE 2
Percent Retention of Reverse-side Impact Strength for
Personal Care/Cosmetics/Perfume Chemicals

| | Copolyester | Copolyester |
|---|---|---|
| Test Chemical | EX 1-A | EX 1-B |
| Coppertone | 85.9 | 83.8 |
| SPF30 sunscreen | | |
| Tanning Spray | | 97 |
| Coty Cologne | | 102.4 |
| Aftershave Gel | | 96.7 |
| Shave Gel | | 92.4 |
| Spray | | 82.2 |
| Antiperspirant | | |
| Deodorant | | |
| Frizz Spray | | 82.8 |
| Foaming Mousse | | 91.7 |
| Hair Masque | | 88.3 |
| Shampoo | | 91.2 |
| Hair Conditioner | | 86 |
| Old Spice Hair | | 88.2 |
| Putty | | |
| Old Spice Styling | | 89.8 |
| Paste | | |
| Old Spice Hair | | 91.5 |
| Pomade | | |
| Anti-aging | | 93.1 |
| Moisturizer | | |
| Tone Perfecting | | 91.8 |
| serum | | |
| Tabac Man EDT | | 89.1 |
| Shine + anti-Frizz | | 86.8 |
| L′Oreal concealer | 109.4 | 108.3 |
| Neutrogena eye | 117.7 | 115.1 |
| makeup remover | | |
| Eucerin original | 94.5 | 97.1 |
| lotion | | |
| Replens eye gel | 112.5 | 109 |
| Australian gold | 93.8 | 88.3 |
| lanolin | | |
| Peppermint | 112.77 | 104.78 |
| Essential Oil | | |
| VS Bombshell | 96.94 | 91.93 |
| Body Mist | | |
| Clinique Super | 91.19 | 85.83 |
| Defense SPF40 | | |
| Caviar Style | 88.85 | 82.87 |
| Concrete | | |
| Tea Tree | 98.92 | 91.1 |
| Essential Oil | | |
| J′Adore Perfume | 97.12 | 91.86 |
| Burt′s Bees | 91.01 | 83.69 |
| Pomegranate Lip | | |
| Balm | | |
| E. Lauder Liquid | 86.87 | 83.69 |
| Found′n Compact | | |

TABLE 3
Percent Retention of Reverse-side Impact Strength for
Chemicals Contacting Wearable Articles

| | Copolyester | Copolyester |
|---|---|---|
| Test Chemical | EX 1-A | EX 1-B |
| Fabreze | 93.2 | 89.9 |
| TIDE original | 94.5 | |
| powder, sat sol′n | | |
| H2O2 | 107.2 | 110.3 |
| 40% DEET | 99.9 | |
| Zeiss wipes | 95.1 | 94.8 |
| Zeiss cleaner | 97 | 102.7 |
| Frosch Vinegar | 95.7 | 93.9 |
| cleaner | | |
| Eclipse cleaning | 96 | 107.4 |
| solution | | |
| Cat Crap anti-fog | 95.1 | 97.8 |

TABLE 4
Percent Retention of Reverse-side Impact Strength for
Solvent Chemicals

| | Copolyester |
|---|---|
| Test Chemical | EX 1-A |
| 2-pyrrolidone | 105.0 |
| 1,2-butanediol | 106.1 |
| 1,2-hexanediol | 106.1 |
| Dowanol | 106.1 |
| 1-(2-Hydroxyethyl)-2- | 98.4 |
| pyrrolidone (HE2P) | |
| methyl ethyl ketone (MEK) | 102.2 |
| ethylhydroxy- | 102.9 |
| propanediol (EHPD) | |
| 100%-2-pyrrolidone | 82.2 |
| Acetone | 95.9 |
| 100%-1,2-hexanediol | 92.6 |
| Ethanol | 97.3 |
| Glycerol | 102.9 |

TABLE 5
Percent Retention of Reverse-side Impact Strength for
Miscellaneous Additional Chemicals

| | Copolyester | Copolyester |
|---|---|---|
| Test Chemical | EX 1-A | EX 1-B |
| Isopropyl alcohol | 80.0 | 83.4 |
| 95% EtOH | 82.4 | 83.6 |
| Simple Green | 91.0 | 91.8 |
| Virex Tb | 89.5 | 86.8 |
| Paclitaxel | 91.0 | 87.3 |
| Docetaxel | 87.9 | 85.9 |
| Clorox Bleach Gel | | 92.3 |
| Cleaner + Bleach | | 93.9 |
| canola oil | 77.9 | 78.1 |
| block cheddar | 78.0 | 78.0 |
| cheese | | |
| whole Milk | 81.6 | 84.2 |
| Ragu traditional | 83.8 | 79.9 |
| spaghetti sauce | | |
| MCT oil (medium | 78.0 | 77.8 |
| chain triglycerides) | | |

A review of Tables 2-5 reveals that both materials had good resistance to all the chemicals tested, with the EX 1-A material generally outperforming the EX 1-B material for the personal care/cosmetics/perfume chemicals, with EX 1-B generally outperforming for the chemicals contacting wearable articles, and with similar performance for the miscellaneous chemicals.

### Comparative Example 1

Similar tests to Example 2 were conducted on test bars made from the following materials: Copolyesters TX1001, TX1501, DX4000, DX4001, EN076 and AN001 (from Eastman Chemical Company); and Cellulose-based engineering bioplastics GC601 1 and GC6021 (from Eastman Chemical Company). The results are shown below in Table 6.

TABLE 6
Percent Retention of Reverse-side Impact Strength for other comparative polymer materials

| Test | | | | | | | | |
|---|---|---|---|---|---|---|---|---|
| Chemical | GC6011 | GC6021 | DX4001 | DX4000 | TX1001 | TX1501 | EN076 | AN001 |
| Coppertone | | | 12.6 | | 26.7 | | | |
| SPF30 | | | | | | | | |
| sunscreen | | | | | | | | |
| Isopropyl | | | 25.9 | | 81.0 | | | |
| alcohol | | | | | | | | |
| 95% EtOH | | | 8.1 | | 94.6 | | | |
| Simple Green | | | 22.0 | | 86.4 | | | |
| Virex Tb | | | 7.6 | | 101.5 | | | |
| canola oil | | | 8.8 | | 78.6 | | | |
| block cheddar | | | 9.3 | | 78.6 | | | |
| cheese | | | | | | | | |
| whole Milk | | | 88.6 | | 88.7 | | | |
| Ragu | | | 10.1 | | 81.0 | | | |
| traditional | | | | | | | | |
| spaghetti | | | | | | | | |
| sauce | | | | | | | | |
| MCT oil | | | 7.7 | | 58.3 | | | |
| (medium | | | | | | | | |
| chain | | | | | | | | |
| triglycerides) | | | | | | | | |
| Tide original | 70.0 | 83.1 | | 8.6 | | 82.8 | | |
| powder, sat. | | | | | | | | |
| sol'n | | | | | | | | |
| 40% DEET | 63.3 | 74.2 | | 6.2 | | 21.5 | | |
| Tanning | | | | 46.1 | | 96.0 | 98.2 | |
| Spray | | | | | | | | |
| Coty Cologne | | | | 7.1 | | 10.4 | 26.5 | |
| Aftershave | | | | 9.6 | | 14.4 | 93.8 | |
| Gel | | | | | | | | |
| Shave Gel | | | | 16.5 | | 91.8 | 95.4 | |
| Fabreze | | | | | | | 94.7 | |
| Frizz Spray | | | | 82.4 | | 87.8 | 94.7 | |
| Foaming | | | | 7.7 | | 62.5 | 51.7 | |
| Mousse | | | | | | | | |
| Hair Masque | | | | 15.6 | | 82.7 | 94.3 | |
| Shampoo | | | | 14.4 | | 89.4 | 96.1 | |
| Hair | | | | 10.0 | | 45.4 | 77.8 | |
| Conditioner | | | | | | | | |
| Old Spice | | | | 44.0 | | 72.5 | 95.0 | |
| Hair Putty | | | | | | | | |
| Clorox Bleach | | | | 3.2 | | 6.0 | 3.4 | |
| Gel | | | | | | | | |
| Old Spice | | | | | | 84.3 | 91.3 | 74.1 |
| Styling Paste | | | | | | | | |
| Old Spice | | | | | | 86.7 | 94.7 | 93.5 |
| Hair Pomade | | | | | | | | |
| Anti-aging | | | | | | 59.8 | 92.4 | 0.0 |
| Moisturizer | | | | | | | | |
| Tone | | | | | | 85.6 | 94.3 | 89.2 |
| Perfecting | | | | | | | | |
| serum | | | | | | | | |
| Tabac Man | | | | | | 7.3 | 3.5 | 7.6 |
| EDT | | | | | | | | |
| Shine + | | | | | | 18.0 | 69.7 | 19.7 |
| anti-Frizz | | | | | | | | |
| Cleaner + | | | | | | 26.3 | 70.5 | 24.1 |
| Bleach | | | | | | | | |
| L'Oreal | 90.8 | 90.5 | | | | | | |
| concealer | | | | | | | | |
| Neutrogena | 95.7 | 90.5 | | | | | | |
| eye makeup | | | | | | | | |
| remover | | | | | | | | |
| Eucerin | 86.6 | 81.8 | | | | | | |
| original lotion | | | | | | | | |
| Replens eye | 94.5 | 92.6 | | | | | | |
| gel | | | | | | | | |
| Australian | 87.2 | 87.8 | | | | | | |
| gold lanolin | | | | | | | | |
| H2O2 | 92.0 | 88.7 | | | | | | |
| Zeiss cleaner | 87.7 | 89.6 | | | | | | |
| Zeiss wipes | 97.1 | 89.6 | | | | | | |
| Frosch | 83.1 | 86.2 | | | | | | |
| vinegar | | | | | | | | |
| cleaner | | | | | | | | |
| Eclipse | 68.9 | 65.0 | | | | | | |
| cleaning | | | | | | | | |
| solution | | | | | | | | |
| Cat Crap | 88.4 | 89.9 | | | | | | |
| anti-fog | | | | | | | | |

Additional tests (similar to Example 2) were conducted for ink formulation chemicals on test bars made from the following materials: Eastalloy P30 (from Eastman Chemical Company); Eastalloy P50 (from Eastman Chemical Company); Eastalloy DA510 (from Eastman Chemical Company); Durastar 1910HF (from Eastman Chemical Company); Copolyester TX1001 (from Eastman Chemical Company); Eastar DN011 (from Eastman Chemical Company); and Eastar GN001 (from Eastman Chemical Company). The results are shown below in Table 7.

TABLE 7
Percent Retention of Reverse-side Impact Strength
for additional comparative polymer materials

| Test | | | | | | | |
|---|---|---|---|---|---|---|---|
| Chemical | P30 | P50 | DA510 | 1910HF | TX1001 | DN011 | GN001 |
| 2-pyrrolidone | 92.3 | 104.1 | 100.3 | 98.7 | 97.7 | 102.0 | 99.1 |
| 1,2-butanediol | 100.9 | 107.4 | 101.5 | 103.7 | 103.8 | 100.0 | 100.8 |
| 1,2-hexanediol | 97.3 | 113.4 | 102.9 | 76.7 | 100.6 | 91.3 | 98.3 |
| Dowanol | 38.7 | 83.6 | 30.2 | 23.3 | 49.5 | 31.6 | 7.5 |
| HE2P | 99.1 | 104.9 | 99.1 | 94.3 | 95.0 | 94.1 | 97.5 |
| MEEG | 74.0 | 71.3 | 53.0 | 31.8 | 72.2 | 52.0 | 20.7 |
| MEK | 104.1 | 54.6 | 101.7 | 27.1 | 102.0 | 99.7 | 37.0 |
| EHPD | 96.2 | 109.0 | 99.4 | 99.7 | 99.3 | 96.7 | 96.6 |
| 100%-2-pyrrolidone | 19.6 | 9.3 | 17.3 | 13.1 | 87.9 | 19.5 | 0.0 |
| Acetone | 92.5 | 105.0 | 97.1 | 83.5 | 95.2 | 101.0 | 99.1 |
| 100%-1,2-hexanediol | 98.0 | 95.3 | 87.7 | 25.5 | 82.5 | 80.5 | 85.2 |
| Ethanol | 103.8 | 108.7 | 99.4 | 101.0 | 103.5 | 94.4 | 98.6 |
| Glycerol | 90.9 | 100.6 | 98.6 | 97.1 | 96.2 | 95.7 | 91.5 |

Additional tests (similar to Example 2) were conducted for 40% DEET and Tide original powder on test bars made from the following materials: PC 2608 (MAKROLON polycarbonate PC2608 from Covestro); Bayblend 85 (Bayblend T85 polycarbonate and ABS blend from Covestro); Bayblend 65 (Bayblend T65 polycarbonate and ABS blend from Covestro); ABS GP 35 (Terluran GP-35 from Ineos); Copolyester TX2001 (from Eastman Chemical Company); and blends of copolyester TX1501 (from Eastman Chemical Company) with ABS GP 22 (Terluran GP-22 from Ineos) with either 15 or 35 wt % ABS. The results are shown below in Table 8.

TABLE 8
Percent Retention of Reverse-side Impact Strength for additional comparative polymer materials

| Test | | Bayblend | Bayblend | | | TX1501/ABS | TX1501/ABS |
|---|---|---|---|---|---|---|---|
| Chemical | PC2608 | 85 | 65 | ABS GP35 | TX2001 | 15% | 35% |
| 40% | 20.6 | 10.0 | 13.9 | 0 | 12.6 | 24.5 | 23.8 |
| DEET | | | | | | | |
| Tide | 46.2 | 58.2 | 59.2 | 23.2 | 81.0 | 92.7 | 93.4 |
| original | | | | | | | |
| powder, | | | | | | | |
| sat. sol'n | | | | | | | |

A review of Tables 6 to 8 reveals that the performance of the comparative materials varied significantly depending on the test chemical used. Also, comparing these tables with Tables 2-5, both the EX 1-A and EX 1-B materials generally outperformed the comparative materials performed more consistently for the various test chemicals used.

Other embodiments will be apparent to those skilled in the art from consideration of the specification and practice of the embodiments disclosed herein. It will be understood that variations and modifications can be affected within the spirit and scope of the disclosed embodiments. It is further intended that the specification and examples be considered as exemplary only, with a true scope and spirit of the disclosed embodiments being indicated by the following claims.

1. An article comprising at least one molded component configured to accommodate contact with a chemical composition that comprises one or more degradation chemicals, wherein said molded component is formed of a copolyester composition comprising at least one copolyester that comprises:
(a) a dicarboxylic acid component comprising:
i) 70 to 100 mole % of terephthalic acid residues;
(b) a glycol component comprising:
i) 5 to 15 mole % of 2,2,4,4-tetramethyl-1,3-cyclobutanediol residues; and
ii) 85 to 95 mole % of 1,4-cyclohexanedimethanol residues,
wherein the total mole % of the dicarboxylic acid component is 100 mole %, and the total mole % of the glycol component is 100 mole %; and wherein the inherent viscosity is 0.60 to 1.2 dL/g as determined in 60/40 (wt/wt) phenol/tetrachloroethane at a concentration of 0.5 g/100 ml at 25° C.; and wherein the polyester has a Tg of 95° C. to 115° C.

2. The article according to claim 1, wherein the copolyester composition has a Tg in the range from 100° C. to 115° C.

3. The article according to claim 2, wherein the copolyester composition has an inherent viscosity is 0.70 to 1.0 dL/g, or 0.75 to 0.95 dL/g.

4. The article according to claim 1, wherein the glycol component comprises:
i) 9 to 13 mole % of 2,2,4,4-tetramethyl-1,3-cyclobutanediol residues; and
ii) 87 to 91 mole % of 1,4-cyclohexanedimethanol residues.

5. The article according to claim 1, wherein the dicarboxylic acid component comprises 100 mole % terephthalic acid residues.

6. The article according to claim 1, wherein the dicarboxylic acid component comprises:
i) 95 to 100 mole % of terephthalic acid residues; and
ii) 0 to 5 mole % of isophthalic acid residues.

7. The article according to claim 6, wherein the dicarboxylic acid component comprises:
i) 97 to 99 mole % of terephthalic acid residues; and
ii) 1 to 3 mole % of isophthalic acid residues.

8. The article according to claim 1, wherein the copolyester composition has a crystallization half time from 30 seconds to 5 minutes.

9. The article according to claim 1, wherein chemical composition is intended for body contact and the article is a wearable article comprising at least one molded component configured to accommodate contact with said chemical composition.

10. An article comprising at least one molded component configured to receive a chemical composition intended for body contact, wherein the chemical composition comprises one or more degradation chemicals, and wherein said molded component is formed of a copolyester composition comprising at least one copolyester that comprises:
(a) a dicarboxylic acid component comprising:
i) 70 to 100 mole % of terephthalic acid residues;
(b) a glycol component comprising:
i) 5 to 15 mole % of 2,2,4,4-tetramethyl-1,3-cyclobutanediol residues; and
ii) 85 to 95 mole % of 1,4-cyclohexanedimethanol residues,
wherein the total mole % of the dicarboxylic acid component is 100 mole %, and the total mole % of the glycol component is 100 mole %; and wherein the inherent viscosity is 0.60 to 1.2 dL/g as determined in 60/40 (wt/wt) phenol/tetrachloroethane at a concentration of 0.5 g/100 ml at 25° C.; and wherein the polyester has a Tg of 95° C. to 115° C.

11. The article according to claim 10, comprising a chemical composition intended for body contact in contact with a surface of said molded component.

12. The article according to claim 11, wherein the chemical composition intended for body contact is in the form of a liquid formulation and/or in the form of a foam or gel.

13. The article according to claim 12, wherein the molded component comprises a container configured to contain a chemical composition intended for body contact and selectively release the chemical composition intended for body contact.

14. The article according to claim 13, wherein the molded component comprises a container configured to contain a chemical composition intended for body contact that is in a liquid formulation form.

15. The article according to claim 12, wherein the molded component comprises a conduit configured to convey a chemical composition intended for body contact.

16. The article according to claim 15, wherein the article comprises one or more molded components that comprise a container configured to contain a chemical composition intended for body contact and a conduit configured to convey a chemical composition intended for body contact, wherein the container and conduit are in fluid communication.

17. The article according to claim 1, wherein the molded component can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles.

18. The article according to claim 1, wherein the plastic composition has a reverse-side impact strength retention of at least 80% after exposure to a terpene oil, when tested according to the methods disclosed in Example 2.

19. The article according to claim 18, wherein the plastic composition has a reverse-side impact strength retention of at least 95% after exposure to d-limonene, when tested according to the methods disclosed in Example 2.

20. The article according to claim 1, wherein the dicarboxylic acid component of the polyester comprises monomer residues having at least 50 mole % recycle content.
